(19) United States
(12) Patent Application Publication
(10) Pub. No.: US 2024/0112764 A1
(43) Pub. Date: Apr. 4, 2024

(54) INFORMATION PROCESSING DEVICE, INFORMATION PROCESSING METHOD, AND NON-TRANSITORY COMPUTER READABLE MEDIUM

(71) Applicants: ENEOS Corporation, Tokyo (JP); Preferred Networks, Inc., Tokyo (JP)

(72) Inventors: Yoshihiro YAYAMA, Tokyo (JP); Yusuke ASANO, Tokyo (JP); Takafumi ISHII, Tokyo (JP); Takao KUDO, Tokyo (JP); Taku WATANABE, Tokyo (JP); Ryohto SAWADA, Tokyo (JP)

(73) Assignees: ENEOS Corporation, Tokyo (JP); Preferred Networks, Inc., Tokyo (JP)

(21) Appl. No.: 18/533,914

(22) Filed: Dec. 8, 2023

Related U.S. Application Data
(63) Continuation of application No. PCT/JP22/23501, filed on Jun. 10, 2022.

(30) Foreign Application Priority Data
Jun. 11, 2021 (JP) 2021-098299

(51) Int. Cl. G16C 20/70

(52) U.S. Cl. CPC G16C 20/70

(57) ABSTRACT

An information processing device includes one or more processors. The one or more processors are configured to optimize, for a specific elementary reaction in a reaction using a catalyst including a plurality of elementary reactions, an arrangement of a promoter element in the catalyst based on activation energy acquired using a trained model, and search for the promoter element based on the activation energy acquired using the trained model for each type of the promoter element.

### CROSS REFERENCE TO RELATED APPLICATIONS

This application is continuation application of International Application No. JP2022/023501, filed on Jun. 10, 2022, which claims priority to Japanese Patent Application No. 2021-098299, filed on Jun. 11, 2021, the entire contents of which are incorporated herein by reference.

### FIELD

This disclosure relates to an information processing device, an information processing method, and a non-transitory computer readable medium.

### BACKGROUND

A catalyst in a chemical reaction is important as a substance for changing the reaction rate. By adding a promoter to the catalyst, the performance of the catalyst increases in some cases. In the field of computational science, Nudged Elastic Band (NEB) by elucidation Density Function Theory (DFT) of a mechanism of the catalytic reaction is sometimes executed. By using this technique, the activation energy of a specific elementary reaction can be calculated. Therefore, it is possible to execute a calculation of the activation energy in the case of adding the promoter by the NEB calculation and estimate the effect of the addition of the promoter.

The reaction rate constant of the elementary reaction can be calculated using the activation energy acquired from the above and theory of absolute reaction rates. Further, it is also possible to calculate the yield of a product obtained by a complex catalytic reaction from the acquired reaction rate constant. By changing the rate constant of each reaction using the above, it is possible to investigate which elementary reaction affects a desired yield.

In this method, it is necessary to search for a promoter which lowers the activation energy for the elementary reaction which increases the desired product yield by promoting the reaction in order to search for a catalyst which improves the performance by adding the promoter. In a prior art, when calculating the activation energy by adding a promoter, for example, a method of arranging a promoter element on a surface of the catalyst, a method of replacing a catalyst element, or the like is used. Therefore, the physical property of the catalyst greatly changes depending on the arrangement place of the promoter, so that the arrangement lower in activation energy is searched for while gradually changing the position, number, and the like of the promoter in the above calculation.

However, the cost of the DFT calculation is very high and the arrangement of the promoter needs to be changed among many atoms as explained above, so that the search for the arrangement of the promoter using this technique is a very difficult operation. If the calculation with the promoter added is executed and an element which lowers the activation energy is found, the improvement in catalytic performance cannot be expected unless the reaction is the elementary reaction which affects the desired product yield.

### BRIEF DESCRIPTION OF DRAWINGS

FIG. 1 is a block diagram illustrating an example of a search device according to an embodiment.

FIG. 2 is a flowchart illustrating processing of the search device according to an embodiment.

FIG. 3 is a flowchart illustrating processing of the search device according to an embodiment.

FIG. 4 is a chart illustrating an example of an outermost surface and an adsorbed molecule according to an embodiment.

FIG. 5 is a diagram illustrating one example of an implementation of an information processing system/device according to an embodiment.

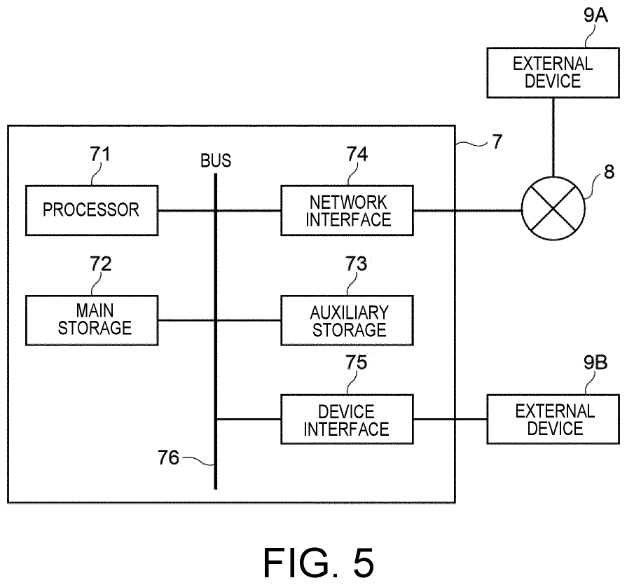

### DETAILED DESCRIPTION

According to an embodiment, an information processing device includes one or more processors. The one or more processors are configured to optimize, for a specific elementary reaction in a reaction using a catalyst including a plurality of elementary reactions, an arrangement of a promoter element in the catalyst based on activation energy acquired using a trained model, and search for the promoter element based on the activation energy acquired using the trained model for each type of the promoter element

This trained model may be a model to be used for Neural Network Potential (NNP) which outputs energy when an atomic structure of a substance is input.

Hereinafter, embodiments of the present invention will be explained with reference to the drawings. The drawings and the explanation of the embodiments are indicated as examples and are not intended to limit the present invention.

### First Embodiment

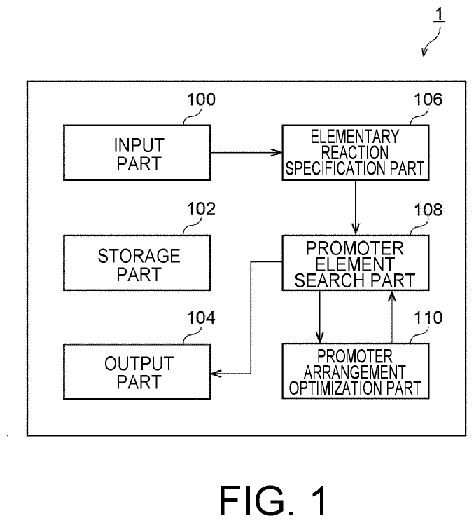

FIG. 1 is a diagram schematically illustrating a search device according to an embodiment. A search device 1 (information processing device) includes an input part 100, a storage part 102, an output part 104, an elementary reaction specification part 106, a promoter arrangement optimization part 110, and a promoter element search part 108. The search device 1 is a device which deduces, when information related to an adsorbed molecule and a catalyst is input, a promoter element which improves the performance by replacing some atoms of the catalyst to a promoter element and/or additionally arranging the promoter element in the catalyst, and an arrangement of the promoter element. Note that a configuration required for the operation of the search device 1 other than the configuration illustrated in FIG. 1 is not illustrated but appropriately provided as necessary.

The input part 100 includes an input interface of the search device 1 and accepts input of data into the search device 1. The search device 1 accepts, for example, information on the adsorbed molecule and the catalyst from a user via the input part 100.

The storage part 102 stores data and the like required for the operation of the search device 1. The storage part 102 stores, for example, information input from the input part 100, a program for causing the search device 1 to operate, and a search result, as well as information on an intermediate value required in the middle of an arithmetic operation, a parameter related to a neural network model to be used for deduction, and so on.

The output part 104 outputs the search result to an external part or the storage part 102. In other words, the output in this disclosure is a concept including output of a result to the storage part 102 of the search device 1 in addition to output to an external storage, monitor, or the like.

The elementary reaction specification part 106 specifies an elementary reaction affecting a desired characteristic (hereinafter, described as a target property) in a plurality of elementary reactions in a catalytic reaction. The elementary reaction specification part 106 may specify an elementary reaction with respect to the target property, for example, by a reaction rate simulation, or may specify an elementary reaction with respect to the target property based on the database obtained by an arithmetic operation, or the like.

The elementary reaction specification part 106 may execute the kinetic simulation while changing a reaction rate constant for each elementary reaction of the plurality of elementary reactions. In this case, the elementary reaction affecting the target property may be specified depending on how the target property has been affected and changed through the catalytic reaction including the elementary reaction with the changed reaction rate constant.

For example, in the case where the time of the catalytic reaction is regarded as the target property, the reaction rate constant may be changed for one or a plurality of elementary reactions, and an elementary reaction having a reaction rate constant which changes the time of the catalytic reaction may be specified. For example, in the case where a yield is regarded as the target property, the reaction rate constant may be changed while keeping an equilibrium constant for each elementary reaction, and an elementary reaction which changes the yield may be specified.

This specification may be, for example, processing of simulating the catalytic reaction while changing the above reaction rate constant for a plurality of elementary reactions and specifying an elementary reaction which has had a greatest effect by the simulation result. More specifically, various reaction rate constants are variously changed for each elementary reaction and the change rate of the target property is calculated, and the highest change rate may be regarded as an influence value of the elementary reaction. Then, the elementary reaction specification part 106 may compare influence values for elementary reactions to specify one or a plurality of elementary reactions from a highest one.

The elementary reaction specification part 106 may execute the kinetic simulation using a later-explained trained model. More specifically, the elementary reaction specification part 106 may calculate a reaction rate parameter of each elementary reaction using the trained model which predicts the potential in the promoter arrangement optimization part 110.

The promoter element search part 108 executes a search for a promoter element lower in activation energy than other promoter elements when desiring to promote a specified elementary reaction based on the activation energy acquired using the trained model for each promoter element. The promoter element search part 108 causes, for example, the promoter arrangement optimization part 110 to execute optimization of the arrangement with the promoter element designated with respect to the adsorbed molecule related to the elementary reaction specified by the elementary reaction specification part 106 and the catalyst. The promoter element search part 108 acquires the information related to the activation energy for each promoter element from the promoter arrangement optimization part 110 and searches for which element is suitable for the specified elementary reaction.

The promoter arrangement optimization part 110 optimizes the arrangement of the promoter element in the catalyst using the trained model for the elementary reaction specified by the elementary reaction specification part 106 and the promoter selected by the promoter element search part. The trained model is a model provided, for example, in NNP (Neural Network Potential), and the promoter arrangement optimization part 110 in this case predicts a potential energy surface near a transition state of each elementary reaction by NNP.

The promoter arrangement optimization part 110 inputs information related to the catalyst in which the promoter element is arranged and the adsorbed molecule to be adsorbed on the catalyst in the target property into the trained model to predict the activation energy. The promoter arrangement optimization part 110, as a non-limiting example, repeatedly executes deduction with various arrangements of the promoter element set in an atomic structure (initial structure in calculation) in an initial state with the position and posture of the admolecule fixed with respect to the catalyst, to acquire information on an arrangement lower in activation energy than other arrangements, thereby optimizing the arrangement of the promoter element.

The promoter arrangement optimization part 110 outputs the activation energy in the optimized arrangement of a target element to the promoter element search part 108.

Note that the trained model is a model trained to be able to predict physical property information using various elements. More specifically, the trained model is a model which receives input of a combination of various elements as an atomic structure and uses appropriate physical property information on energy with respect to the atomic structure as training data to optimize a parameter based on an error between an output value of the model and training data. Further, the trained model may be in a form capable of receiving input of a boundary condition as the atomic structure. In this case, the trained model may be in a form capable of designating the atomic structure being a unit, a pitch being a repetition of the unit, and whether it is periodic or free space. In other words, the trained model may be the one trained as a model which can receive input as that of general NNP. The input into the trained model may be an element and coordinates (position) of each of atoms constituting a substance, and the output may be potential (energy) or information required for calculating energy such as a wave function.

The trained model may be a model which has been trained using a result related to potential, for example, by a DFT calculation or other first-principles calculation, as training data.

In the above, the trained model is assumed to be able to acquire the activation energy by NNP but, not limited to this, may be, for example, a model which acquires a physical property value correlated/highly correlated with the activation energy. Examples of the physical property value include an intermolecular distance, an atomic charge, adsorption energy, a vibration frequency, a d band center, and energy of a reactive intermediate. The trained model may be a model which predicts at least one piece of information among them with respect to the atomic structure. In this case, the promoter arrangement optimization part 110 acquires the arrangement of the promoter element indicating a lower physical property value (or a higher physical property value when there is a negative correlation) than those of other promoter elements based on these physical property values.

The promoter element search part 108 determines an element suitable as the promoter element by using the physical property value output from the promoter arrangement optimization part 110, and outputs it via the output part 104. In the case where the promoter arrangement optimization part 110 outputs the value of the activation energy, the promoter element search part 108 selects and outputs a promoter element lower or higher in activation energy than the other promoter elements. In the case of using a physical property value correlated to the activation energy as the physical property value, the promoter element is selected based on the physical property value and output.

The promoter element search part 108 may cause the promoter arrangement optimization part 110 to execute optimization using the promoter element as one type of element. In this case, the promoter element search part 108 designates one type of element as the promoter element, and causes the promoter arrangement optimization part 110 to execute optimization. The promoter arrangement optimization part 110 executes optimization of the arrangement of the promoter element designated by the promoter element search part 108. The promoter element search part 108 changes the promoter element to various types of elements, and causes the promoter arrangement optimization part 110 to repeatedly execute optimization, thereby executing the search for a promoter element.

The promoter element search part 108 may cause the promoter arrangement optimization part 110 to execute optimization with a plurality of types of elements designated as the promoter element. In this case, the promoter element search part 108 causes the promoter arrangement optimization part 110 to execute optimization with a plurality of types of elements as the promoter element. The promoter arrangement optimization part 110 executes optimization of the arrangement of the promoter element designated by the promoter element search part 108. The promoter element search part 108 changes the promoter element to various combinations of a plurality of elements, and causes the promoter arrangement optimization part 110 to repeatedly execute optimization, thereby executing the search for a promoter element.

Further, in the case where a plurality of types of promoter elements are designated, the promoter arrangement optimization part 110 may optimize the arrangement of the plurality of types of promoter elements and optimize the ratio of arrangement of the plurality of types of promoter elements.

In common with the above, the promoter arrangement optimization part 110 repeats a reaction path search using the trained model a plurality of times while designating the arrangement of one or a plurality of types of promoter elements designated by the promoter element search part 108, acquires during this time the activation energy or the physical property value correlated with the activation energy, and optimizes the arrangement of the promoter element based on the value.

The promoter arrangement optimization part 110 arranges one of a plurality of promoter elements designated by the promoter element search part 108 in the catalyst, and executes the above optimization. In the case of optimizing the arrangement of one promoter element, the promoter arrangement optimization part 110 may execute optimization by a grid search as a non-limiting example. Besides, in the case of optimizing the arrangement of a plurality of promoter elements, the promoter arrangement optimization part 110 may execute optimization by Bayesian optimization or a random search as a non-limiting example.

Further, the promoter arrangement optimization part 110 may set the distance between the catalyst and the adsorbed molecule to within 5 Å as the initial structure in the above calculation. Further, the promoter arrangement optimization part 110 may set the distance between the adsorbed molecule and the promoter element to be arranged also to within 5 Å as the initial structure.

As explained above, the promoter arrangement optimization part 110 may arrange the promoter element by replacing one or a plurality of atoms in the atomic structure constituting the catalyst with the promoter element, or may arrange the promoter element by adding one or a plurality of promoter elements to the atomic structure constituting the catalyst. The number of promoter elements arranged may not contribute to the improvement in reaction rate if it is too large or too small. Therefore, the promoter arrangement optimization part 110 may arrange, for example, less than 10% of atoms in number in a catalyst atomic structure other than the adsorbed molecule to be input into the trained model, as the promoter elements. This number of atoms of 10% is exemplified as a non-limiting example. If too many promoter elements are input into the atomic structure constituting the catalyst in calculation, a surface structure becomes unstable depending on the element type, and the surface structure may greatly change as compared with the case without promoter. In order to avoid the change in surface structure, the number of atoms is set to less than 10%.

The elementary reaction specification part 106 can also specify a plurality of elementary reactions as an elementary reaction large in percentage constituting to the target property. The same processing may be executed for different elementary reactions after the promoter element search part 108 searches for a promoter element for a certain elementary reaction as explained above. The promoter element search part 108 may execute a search for a promoter element in order of the elementary reaction determined to be larger in contributing percentage by the elementary reaction specification part 106.

In this case, the promoter element search part 108 may store a search result, for example, in the storage part 102, and execute a search for a promoter element in the next elementary reaction using the search result. Then, the promoter element search part 108 may collectively determine stored reaction energy, reaction rate, yield, and the like with respect to a plurality of elementary reactions, and output a final promoter element and the arrangement of the promoter element.

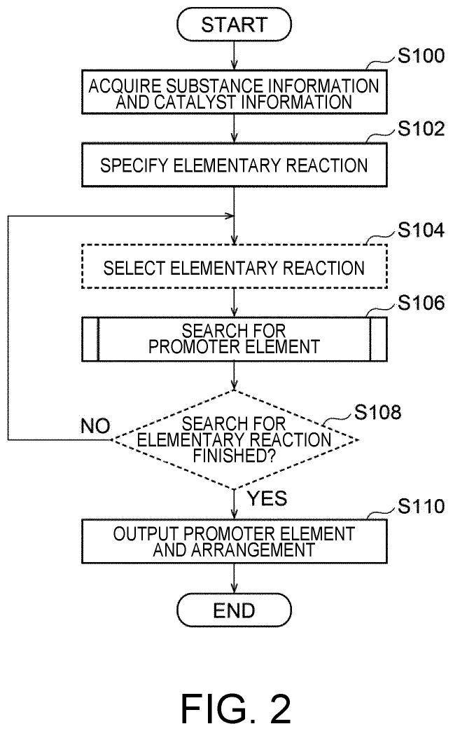

FIG. 2 is a flowchart illustrating processing of the search device 1 according to an embodiment.

The search device 1 acquires information related to a substance to be searched for and a catalyst via the input part 100 (S100). The acquired information may be stored in the storage part 102. The information related to the substance may be input as a reaction formula.

The elementary reaction specification part 106 specifies the elementary reaction which affects the target property in the combination of the substance and the catalyst (S102). In the case of inputting the information related to the substrate in the reaction formula, the elementary reaction specification part 106 specifies a reaction, from which intermediate state to which intermediate state among intermediate states in the reaction path, affecting the target property. As the target property, at least one of property of a reaction rate, a reaction time, and a yield may be designated as a non-limiting example. As a concrete example, the elementary reaction specification part 106 may specify the elementary reaction affecting the yield of a product. Further, as another example, the elementary reaction specification part 106 may specify the elementary reaction affecting the durability of the catalyst.

For example, in the case where the reaction rate of a solid catalyst is regarded as the target property, the elementary reaction is specified from the following equation.

$${k}_{ads} = \frac{{P}^{0} A}{\sqrt{2 π m {k}_{b} T}} \qquad ( 1 )$$

$${k}_{des} = \frac{{k}_{ads}}{{K}_{eq}} = \frac{{P}^{0} A}{\sqrt{2 π m {k}_{b} T}} \frac{exp ( {S}_{gas} / R )}{{q}_{vib , ads}} exp ( \frac{Δ {H}_{ads}^{corr} - {H}_{gas}^{298.15 \to T} + {E}_{lat}}{RT} ) \qquad ( 2 )$$

$${k}_{surf} = \frac{{k}_{b} T}{h} \frac{{Q}^{‡}}{Q} exp ( - \frac{Δ {E}_{act}^{zpe}}{{k}_{b} T} ) \qquad ( 3 )$$

$$Q = {\prod }_{i} \frac{1}{1 - exp ( - \frac{h {ω}_{i}}{2 π {k}_{b} T} )} \qquad ( 4 )$$

Here, k represents a rate constant. Besides, k_{b} represents a Boltzmann constant, T represents an absolute temperature, m represents a mass of gas molecule, P⁰ represents a standard pressure 1 atm, A represents a catalyst surface area, K_{eq} represents an equilibrium constant, R represents a gas constant, S_{gas} represents gas entropy, ΔH_{ads}^{corr} represents adsorption energy, H_{gas}^{298->T} represents a change from 298K of gas enthalpy, Eₗₐₜ represents a correcting term expressing interaction between adsorbed molecule, q_{vib,ads} represents a term introduced because all entropy is not lost when a molecule is adsorbed, Q^{∓} represents a partition function in a transition state, and h represents a Plank's constant. Subscripts represent ads: adsorption, des: desorption, and surf: surface. The elementary reaction specification part 106 calculates rate constants at adsorption, desorption, and surface based on Equation (1) to Equation (3). The activation energy ΔE_{act}^{zpe} is calculated based on DFT, and values of the entropy S and the enthalpy H are acquired using the database. The calculation is made with q_{vib, ads} as 1. The partition function Q is acquired by calculating an eigenvalue ωᵢ² with and an angular frequency ωᵢ in a Hessian matrix by a vibration calculation of DFT according to Equation (4). The DFT calculation may be performed by an arithmetic operation by NNP using the trained model.

Then, the rate constant k is converted into the reaction rate r according to the following equation, and the reaction rate r is summed for each element to acquire a time differentiation of concentration.

$${r}_{ads}^{surf} = \frac{{N}_{surf}}{{N}_{total}} \times {( {θ}_{*}^{surf} )}^{n} \times {a}_{gas} \times {k}_{ads}^{surf} \qquad ( 5 )$$

$${r}_{des}^{surf} = \frac{{N}_{surf}}{{N}_{total}} \times {( {θ}_{ads}^{surf} )}^{n} \times {k}_{des}^{surf} \qquad ( 6 )$$

$${r}_{surf} = {k}_{surf} {\prod }_{i} {θ}_{i}^{{v}_{i , surf}} \qquad ( 7 )$$

$$\frac{d {θ}_{i}}{dt} = {\sum }_{j} {v}_{i , j} {r}_{j} \qquad ( 8 )$$

Here, r_{ads}^{surf} represents a reaction rate of an absorption reaction, r_{des}^{surf} represents a reaction rate of a desorption reaction, r_{surf} represents a reaction rate of a surface reaction, r^{j} represents a reaction rate of a reaction j, N_{surf}/Nₜₒₜₐₗ represents a percentage of a specific surface site, θ*^{surf} represents a coverage of an empty site, θ_{ads}^{surf} represents a coverage of the adsorbed molecule, θᵢ represents a coverage of chemical species i, v_{i, surf} represents a stoichiometric coefficient of chemical species i, v_{i,j} represents a stoichiometric coefficient of a reaction j of chemical species i, a_{gas} represents a ratio of a gas partial pressure P/standard pressure P⁰, n represents a coefficient of 2 in the case of dissociation adsorption and 1 otherwise, k_{ads}^{surf} represents a rate constant of absorption, and k_{des}^{surf} represents a rate constant of desorption. Further, the yield of each substance can be acquired by solving simultaneous ordinary differential equations.

For specifying the elementary reaction, for example, DRC (Degree of Rate Control), DSC (Degree of Selectivity Control), DCGC (Degree of Chain-Growth Control) are used. Each of them is given in the following equation. In the following equation, an example of dissociating CO to produce CH₄ is described, and this reaction can be changed by a reaction desired to be executed.

$$D R {C}_{CO}^{i} = {( \frac{\partial ln {r}_{CO}}{\partial ln {k}_{i}} )}_{{k}_{j \neq i} , {K}_{i}} \qquad ( 9 )$$

$$D S {C}_{{CH}_{4}}^{i} = {S}_{{CH}_{4}} \cdot ( D R {C}_{{CH}_{4}}^{i} - D R {C}_{CO}^{i} ) \qquad ( 10 )$$

$$D C G {C}^{i} = {( \frac{\partial α}{\partial ln {k}_{i}} )}_{{k}_{j \neq i} , {K}_{i}} \qquad ( 11 )$$

Here, i represents a reaction i, r_{CO} represents a CO molecular reaction rate, kᵢ represents a rate constant of the reaction i, S_{CH4} represents a selectivity of CH₄, a represents a chain growth probability, and Kᵢ represents a reaction i equilibrium constant.

In an actual catalytic reaction, which elementary reaction is subjected to rate controlling in the whole reaction is discussed but it is difficult to answer this discussion. More specifically, a plurality of late elementary reactions are sometimes present. By performing the reaction rate simulation with the reaction rate constant of each elementary reaction changed using the activation energy of the elementary reaction acquired from the DFT calculation or the database, it is possible to evaluate how much the elementary reaction affects the product yield and/or the chain growth probability. The use of this technique can specify a plurality of elementary reactions affecting the yield and/or the chain growth probability.

The elementary reaction specification part 106 specifies one or a plurality of elementary reactions contributing to the target property from the above relation.

When the elementary reaction specification part 106 specifies a plurality of elementary reactions, the promoter element search part 108 selects one of the specified elementary reactions (S104), and searches for a promoter element (S106). Note that if the specified elementary reaction is one, the processing at S104 is not necessary.

The promoter element search part 108 determines whether the search for the other elementary reaction has been finished after the finish of the search for the type of the promoter element for the one elementary reaction and the arrangement of the promoter element (S108). This determination can also be omitted if the specified elementary reaction is one, as at S104.

If the search for each elementary reaction has not been finished (S108: NO), an elementary reaction other than the elementary reaction for which the search has been finished is selected (S104), and the search processing is continued. If the search for the specified elementary reaction has been finished (S108: YES), the promoter element search part 108 outputs the promoter element and the arrangement (S110), and finishes the processing. In the case where a plurality of elementary reactions are specified, the promoter element search part 108 appropriately updates a promoter element and its arrangement based on the search result of the promoter element for the plurality of elementary reactions, and outputs the search result.

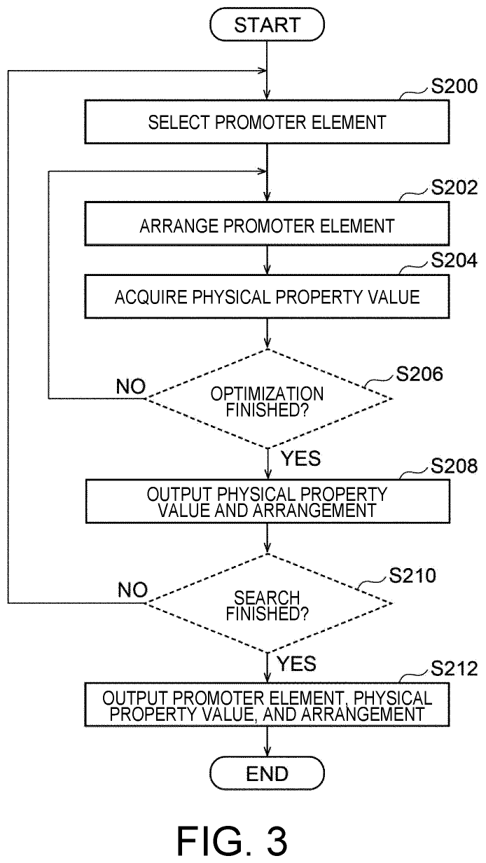

FIG. 3 is a flowchart illustrating search processing for the promoter element in FIG. 2. The processing at S106 in FIG. 2 will be explained using FIG. 3.

First, the promoter element search part 108 selects a promoter element (S200). For example, in the case of arranging one type of promoter element in the catalyst, the promoter element search part 108 selects one from elements which can be applied as the promoter element and transmits it to the promoter arrangement optimization part 110 and transmits a request for optimization of the arrangement. In the case of arranging a plurality of types of promoter elements, the promoter element search part 108 selects a plurality of types of elements from elements which can be applied as the promoter elements and transmits a request for optimization of the arrangement.

The promoter arrangement optimization part 110 arranges the promoter element with respect to the catalyst in order to optimize the arrangement of the promoter element based on the information on the elementary reaction, the catalyst, and the promoter element received from the promoter element search part 108 (S202).

After the arrangement of the promoter element, the promoter arrangement optimization part 110 acquires a physical property value in this arrangement (S204). For example, this physical property value is activation energy in the elementary reaction.

The promoter arrangement optimization part 110 acquires the activation energy based on the information in the catalyst on which the promoter element is arranged and the adsorbed molecule, for example, using the NEB method.

The promoter arrangement optimization part 110 sets, as a non-limiting example, an initial state and a final state of the chemical reaction formula of the elementary reaction, and acquires the activation energy by the optimization calculation using the NEB method. The NEB method appropriately selects an initial path from an initial state (IS) structure to a final state (FS) structure and executes optimization so that the activation energy in this transition becomes low. The promoter arrangement optimization part 110 uses NNP by the trained model and can thereby speedily acquire the energy value in each transition state of the path required for the optimization calculation.

The promoter arrangement optimization part 110 can also acquire, as a non-limiting example, energy in a transition state by a technique using optimization of a transition state (TS) structure and IRC (Intrinsic Reaction Coordinate) using the TS structure acquired by the NEB method. This technique is a technique of preparing the initial structure close to the transition state, calculating a quadratic differential of energy of the initial structure with respect to the structure to acquire a reference vibration, and confirming a virtual vibration mode to search for the transition state (TS) structure. The IRC calculation is a technique of executing a normal structure optimization in a reaction coordinate direction from the TS structure to confirm whether it converges to a structure of a target reactant (IS) and a product (FS). Also in this technique, the use of NNP by the trained model in the processing for finding energy makes it possible to speedily execute the search.

For example, the promoter arrangement optimization part 110 executes vibrational analysis on the TS structure acquired by the NEB calculation with the catalyst, the outermost layer of the promoter element, and the adsorbed molecule set as targets of the arithmetic operation. Then, the promoter arrangement optimization part 110 executes optimization by the Broyden-Fletcher-Goldfarb-Shanno (BFGS) method using the vibration states adjacent to the virtual vibration of the TS structure.

An example of the dissociation reaction of CO with Co regarded as the catalyst and CO regarded as the adsorbed molecule will be explained. The promoter arrangement optimization part 110 extracts one Co atom present at the periphery of CO, and replaces the Co atom with the promoter element. Then, the promoter arrangement optimization part 110 acquires the activation energy using the optimization of the TS structure and IRC.

After the acquisition of the activation energy, the promoter arrangement optimization part 110 determines whether the optimization has been finished (S206). For example, the promoter arrangement optimization part 110 may determine whether the optimization has been finished depending on whether the extraction from the Co atom present at the periphery of CO has been finished.

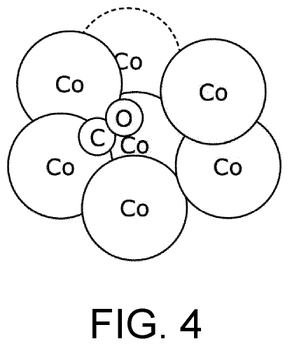

FIG. 4 is a chart illustrating an example of the outermost surface of the catalyst Co and the initial structure of the adsorbed molecule CO. In this chart, the promoter arrangement optimization part 110 selects, for example, the Co atoms illustrated by solid lines one by one and replaces it with the promoter element (S202), and calculates the activation energy by the above technique (S204). The above is repeated for the Co atom illustrated by a solid line to be replaced with the promoter element (from S206: NO to S202, S204). The extraction of all the Co atoms is finished, the promoter arrangement optimization part 110 determines that the optimization of the arrangement has been finished (S206: YES), and outputs the physical property value and the arrangement (S208). For example, in the case of the atomic structure of the surface as illustrated in FIG. 4, an atom to be replaced may be selected from six atoms of the catalyst. This arrangement is exemplified as one example, and may differ depending on the atomic arrangement of the catalyst.

In the case of additionally arranging the promoter element on the surface of Co, the activation energy is acquired with the position of addition designated, and the above arithmetic operation is executed (S202 to S208). Similarly, in the case of arranging a plurality of types of promoter elements, a combination of Co atoms is selected and replaced with a combination of promoter elements or a combination of promoter elements is added to the outermost surfaces of the Co atoms, and the above arithmetic operation is executed (S202 to S208).

These processes can be appropriately performed in parallel. For example, the use of a Graphics Processing Unit (GPU) enables execution of NNP arithmetic calculations for a plurality of atomic structures in parallel. Therefore, it is possible to acquire physical property values in these plurality of arrangements in parallel arithmetic operations, for example, after designation of the plurality of arrangements at S202. Further, for a plurality of promoter elements or the like, arranging the plurality of promoter elements at S202 and subjecting them to parallel processing makes it possible to perform arithmetic operations of optimization of the arrangement of the plurality of promoter elements in parallel. An accelerator to be used is not limited to GPU but may be hardware of another appropriate architecture.

In the above method, the activation energy can be E_{act, forward}=E_{TS}−E_{IS} in the forward direction, and E_{act, backward}=E_{TS}−E_{FS} in the backward direction. E_{TS} is energy in the transition state, and E_{Is} and E_{FS} are each energy in each of the initial state and the final state. The use of NNP for the calculation of the energy makes it possible to speedily execute the arithmetic operation.

Note that if the distance between the adsorbed molecule and the promoter element is too large, it becomes difficult to acquire the effect as the promoter. Therefore, in order to appropriately execute the optimization operation in a real time, the distance between the adsorbed molecule and the promoter element may be set to within 5 Å as the initial structure. Desirably, the distance between the adsorbed molecule and the promoter element may be set to within 4 Å.

In the case of designating the distance, the promoter arrangement optimization part 110 may execute optimization for the catalyst atom within a predetermined distance from the adsorbed molecule irrespective of the atom illustrated by a solid line in FIG. 4. In this case, the number of combinations may be large. Hence, the promoter arrangement optimization part 110 may execute the arrangement of the promoter element to be replaced or added by various optimization techniques.

Though NEB, and the optimization of the TS structure and IRC are used in the above, these techniques can be arbitrarily decided. For example, only the NEB method may be used, only the optimization of the TS structure and IRC may be used, or another appropriate technique may be used for execution.

In the case of arranging one promoter element, the promoter arrangement optimization part 110 may perform optimization, for example, by a grid search. In the case of arranging a plurality of promoter elements, the promoter arrangement optimization part 110 may use, for example, Bayesian optimization, a random search, or a genetic algorithm. These are exemplified as non-limiting examples.

The optimization of the arrangement of a promoter element (or a combination of promoter elements, hereinafter, collectively described as a promoter element or the like) is finished, the promoter element search part 108 determines whether the search for the promoter element has been finished (S210). This determination determines whether the optimization of the arrangement has been finished for the element to be a candidate of the promoter element, or whether the optimization of the arrangement has been finished for an appropriate combination in the case of a plurality of types of promoter elements.

If the optimization of the arrangement for the promoter element or the like has not been finished (S210: NO), the promoter element or the like for which the arrangement has not been optimized is selected (S200), and the processing from S202 is repeated. The promoter arrangement optimization part 110 executes the optimization of the arrangement by changing the promoter element or the like without changing the initial arrangement (initial atomic structure) of the catalyst and the adsorbed molecule. By not changing the initial arrangement, it becomes possible to acquire the physical property value of the activation energy or the like under the same condition.

If the optimization of the arrangement for the promoter element or the like has been finished (S210: YES), the promoter element search part 108 outputs the required information such as the best physical property value, for example, the promoter element whose activation energy is acquired, the physical property value, and the arrangement of the promoter element, and finishes the processing (S212).

As explained above, according to this embodiment, it becomes possible to appropriately search for the promoter element in the catalytic reaction using NNP. The appropriate use of NNP makes it possible to speedily search for a promoter element which improves, for example, the target propergy in the catalytic reaction, for example, the yield of a product and the arrangement of the promoter element.

### Second Embodiment

In the above first embodiment, the search device 1 aims to search for a promoter element which improves a target property such as the yield of a product and its arrangement using the trained model. The search device 1 may be in a form of performing training by active learning so as to further increase the search efficiency and execute acquisition of a new promoter element.

For example, the promoter element search part 108 may execute the active learning of a model which acquires the activation energy using a property amount obtained from the type and the arrangement of the promoter element and data on the activation energy acquired during the search for the promoter element. The model which acquires the activation energy is, for example, a regression model.

The promoter arrangement optimization part 110 acquires various promoter elements and data on the activation energy about their arrangements in the search for the promoter element. The promoter element search part 108 executes the active learning of the regression model using the various promoter elements and the data on the activation energy.

The promoter arrangement optimization part 110 trains the regression model to output the activation energy when inputting the promoter element with respect to the catalyst, for example, using an arbitrary appropriate machine learning technique. The use of the model generated by the training makes it possible to more speedily acquire what promoter element is used with respect to the catalyst to make the activation energy low or high. Note that the input into the regression model may be only the type of the promoter element or the type and the arrangement of the promoter element other than the catalyst and, not limited to them, another value acquired by the promoter arrangement optimization part 110 may be regarded as the input value.

According to this embodiment, the search device 1 executes the search and can generate the regression model which acquires the activation energy for the promoter element, so that the use of the regression model makes it possible to realize the search for a more efficient promoter element.

In each of the above embodiments, some implementation examples will be given. The technique used for the search device 1 is not limited to these examples.

The search device 1 may acquire a required value of the DFT calculation value of the activation energy from a literature value or the database to specify the elementary reaction in the elementary reaction specification part 106.

The search device 1 may acquire a required value using an NNP calculation to specify the elementary reaction in the elementary reaction specification part 106. As explained above, the elementary reaction specification part 106 can also acquire a parameter related to a kinetic simulation in each elementary reaction by the NNP calculation. For example, as values to be used in the kinetic simulation in addition to Q in Equation (4), the elementary reaction specification part 106 can deduce various parameters using the trained model to be used by the promoter arrangement optimization part 110.

The search device 1 may search for the promoter element, for example, using the elementary reaction specified by the user. In this case, the elementary reaction specification part 106 does not need to be provided in the search device 1, and the search device 1 may accept input of the elementary reaction at S100 without executing the processing at S102 in FIG. 2.

The search device 1 may use the element substitution or the element addition by the Bayesian optimization or the element substitution or the element addition by the random search in the promoter arrangement optimization part 110. As another example, the grid search or the genetic algorithm may be used. The percentage of the arrangement can be arbitrary, and may be defined, for example, in a range of 10% or less. The promoter arrangement optimization part 110 may acquire the physical property value from a technique such as the NEB method, the TS structure optimization+IRC, and an arbitrary appropriate combination of these techniques. The physical property value may be the activation energy or a physical property value having a positive correlation or a negative correlation with the activation energy.

The search device 1 may select an element low in activation energy in the case of desiring to promote the specified elementary reaction in the promoter element search part 108. Further, the promoter element search part 108 may select an element so that a physical property value having a positive correlation with the activation energy becomes low or a physical property value having a negative correlation with the activation energy becomes high.

In each of the above embodiments, the search device 1 may be implemented by one or more computers. For example, the input may be in such a form that a client on the user side may perform the input and required information may be transmitted from the client to the search device 1. This case may have such a form that the search device 1 may be provided as a server being a part of a search system.

The trained models of above embodiments may be, for example, a concept that includes a model that has been trained as described and then distilled by a general method.

Some or all of each device (the information processing device or an information processing device which is belonging to the information processing system 1) in the above embodiment may be configured in hardware, or information processing of software (program) executed by, for example, a CPU (Central Processing Unit), GPU (Graphics Processing Unit). In the case of the information processing of software, software that enables at least some of the functions of each device in the above embodiments may be stored in a non-volatile storage medium (non-volatile computer readable medium) such as CD-ROM (Compact Disc Read Only Memory) or USB (Universal Serial Bus) memory, and the information processing of software may be executed by loading the software into a computer. In addition, the software may also be downloaded through a communication network. Further, entire or a part of the software may be implemented in a circuit such as an ASIC (Application Specific Integrated Circuit) or FPGA (Field Programmable Gate Array), wherein the information processing of the software may be executed by hardware.

A storage medium to store the software may be a removable storage media such as an optical disk, or a fixed type storage medium such as a hard disk, or a memory. The storage medium may be provided inside the computer (a main storage device or an auxiliary storage device) or outside the computer.

FIG. 6 is a block diagram illustrating an example of a hardware configuration of each device (the information processing device or the information processing device which is belonging to the information processing system 1) in the above embodiments. As an example, each device may be implemented as a computer 7 provided with a processor 71, a main storage 72 (a main storage device 72), an auxiliary storage 73 (a auxiliary storage device 73), a network interface 74, and a device interface 75, which are connected via a bus 76.

The computer 7 of FIG. 6 is provided with each component one by one but may be provided with a plurality of the same components. Although one computer 7 is illustrated in FIG. 6, the software may be installed on a plurality of computers, and each of the plurality of computer may execute the same or a different part of the software processing. In this case, it may be in a form of distributed computing where each of the computers communicates with each of the computers through, for example, the network interface 74 to execute the processing. That is, each device (the information processing device or the information processing device which is belonging to the information processing system 1) in the above embodiments may be configured as a system where one or more computers execute the instructions stored in one or more storages to enable functions. Each device may be configured such that the information transmitted from a terminal is processed by one or more computers provided on a cloud and results of the processing are transmitted to the terminal.

Various arithmetic operations of each device (the information processing device or the information processing device which is belonging to the information processing system 1) in the above embodiments may be executed in parallel processing using one or more processors or using a plurality of computers over a network. The various arithmetic operations may be allocated to a plurality of arithmetic cores in the processor and executed in parallel processing. Some or all the processes, means, or the like of the present disclosure may be implemented by at least one of the processors or the storage devices provided on a cloud that can communicate with the computer 7 via a network. Thus, each device in the above embodiments may be in a form of parallel computing by one or more computers.

The processor 71 may be an electronic circuit (such as, for example, a processor, processing circuitry, processing circuitry, CPU, GPU, FPGA, or ASIC) that executes at least controlling the computer or arithmetic calculations. The processor 71 may also be, for example, a general-purpose processing circuit, a dedicated processing circuit designed to perform specific operations, or a semiconductor device which includes both the general-purpose processing circuit and the dedicated processing circuit. Further, the processor 71 may also include, for example, an optical circuit or an arithmetic function based on quantum computing.

The processor 71 may execute an arithmetic processing based on data and/or a software input from, for example, each device of the internal configuration of the computer 7, and may output an arithmetic result and a control signal, for example, to each device. The processor 71 may control each component of the computer 7 by executing, for example, an OS (Operating System), or an application of the computer 7.

Each device (the information processing device or the information processing device which is belonging to the information processing system 1) in the above embodiments may be enabled by one or more processors 71. The processor 71 may refer to one or more electronic circuits located on one chip, or one or more electronic circuitries arranged on two or more chips or devices. In the case of a plurality of electronic circuitries are used, each electronic circuit may communicate by wired or wireless.

The main storage device 72 may store, for example, instructions to be executed by the processor 71 or various data, and the information stored in the main storage device 72 may be read out by the processor 71. The auxiliary storage device 73 is a storage device other than the main storage device 72. These storage devices shall mean any electronic component capable of storing electronic information and may be a semiconductor memory. The semiconductor memory may be either a volatile or non-volatile memory. The storage device for storing various data or the like in each device (the information processing device or the information processing device which is belonging to the information processing system 1) in the above embodiments may be enabled by the main storage device 72 or the auxiliary storage device 73 or may be implemented by a built-in memory built into the processor 71. For example, the storages in the above embodiments may be implemented in the main storage device 72 or the auxiliary storage device 73.

In the case of each device (the information processing device or the information processing device which is belonging to the information processing system 1) in the above embodiments is configured by at least one storage device (memory) and at least one of a plurality of processors connected/coupled to/with this at least one storage device, at least one of the plurality of processors may be connected to a single storage device. Or at least one of the plurality of storages may be connected to a single processor. Or each device may include a configuration where at least one of the plurality of processors is connected to at least one of the plurality of storage devices. Further, this configuration may be implemented by a storage device and a processor included in a plurality of computers. Moreover, each device may include a configuration where a storage device is integrated with a processor (for example, a cache memory including an L1 cache or an L2 cache).

The network interface 74 is an interface for connecting to a communication network 8 by wireless or wired. The network interface 74 may be an appropriate interface such as an interface compatible with existing communication standards. With the network interface 74, information may be exchanged with an external device 9A connected via the communication network 8. Note that the communication network 8 may be, for example, configured as WAN (Wide Area Network), LAN (Local Area Network), or PAN (Personal Area Network), or a combination of thereof, and may be such that information can be exchanged between the computer 7 and the external device 9A. The internet is an example of WAN, IEEE802.11 or Ethernet (registered trademark) is an example of LAN, and Bluetooth (registered trademark) or NFC (Near Field Communication) is an example of PAN.

The device interface 75 is an interface such as, for example, a USB that directly connects to the external device 9B.

The external device 9A is a device connected to the computer 7 via a network. The external device 9B is a device directly connected to the computer 7.

The external device 9A or the external device 9B may be, as an example, an input device. The input device is, for example, a device such as a camera, a microphone, a motion capture, at least one of various sensors, a keyboard, a mouse, or a touch panel, and gives the acquired information to the computer 7. Further, it may be a device including an input unit such as a personal computer, a tablet terminal, or a smartphone, which may have an input unit, a memory, and a processor.

The external device 9A or the external device 9B may be, as an example, an output device. The output device may be, for example, a display device such as, for example, an LCD (Liquid Crystal Display), or an organic EL (Electro Luminescence) panel, or a speaker which outputs audio. Moreover, it may be a device including an output unit such as, for example, a personal computer, a tablet terminal, or a smartphone, which may have an output unit, a memory, and a processor.

Further, the external device 9A or the external device 9B may be a storage device (memory). The external device 9A may be, for example, a network storage device, and the external device 9B may be, for example, an HDD storage.

Furthermore, the external device 9A or the external device 9B may be a device that has at least one function of the configuration element of each device (the information processing device or the information processing device which is belonging to the information processing system 1) in the above embodiments. That is, the computer 7 may transmit a part of or all of processing results to the external device 9A or the external device 9B, or receive a part of or all of processing results from the external device 9A or the external device 9B.

In the present specification (including the claims), the representation (including similar expressions) of “at least one of a, b, and c” or “at least one of a, b, or c” includes any combinations of a, b, c, a-b, a-c, b-c, and a-b-c. It also covers combinations with multiple instances of any element such as, for example, a-a, a-b-b, or a-a-b-b-c-c. It further covers, for example, adding another element d beyond a, b, and/or c, such that a-b-c -d.

In the present specification (including the claims), the expressions such as, for example, “data as input,” “using data,” “based on data,” “according to data,” or “in accordance with data” (including similar expressions) are used, unless otherwise specified, this includes cases where data itself is used, or the cases where data is processed in some ways (for example, noise added data, normalized data, feature quantities extracted from the data, or intermediate representation of the data) are used. When it is stated that some results can be obtained “by inputting data,” “by using data,” “based on data,” “according to data,” “in accordance with data” (including similar expressions), unless otherwise specified, this may include cases where the result is obtained based only on the data, and may also include cases where the result is obtained by being affected factors, conditions, and/or states, or the like by other data than the data. When it is stated that “output/outputting data” (including similar expressions), unless otherwise specified, this also includes cases where the data itself is used as output, or the cases where the data is processed in some ways (for example, the data added noise, the data normalized, feature quantity extracted from the data, or intermediate representation of the data) is used as the output.

In the present specification (including the claims), when the terms such as “connected (connection)” and “coupled (coupling)” are used, they are intended as non-limiting terms that include any of “direct connection/coupling,” “indirect connection/coupling,” “electrically connection/coupling,” “communicatively connection/coupling,” “operatively connection/coupling,” “physically connection/coupling,” or the like. The terms should be interpreted accordingly, depending on the context in which they are used, but any forms of connection/coupling that are not intentionally or naturally excluded should be construed as included in the terms and interpreted in a non-exclusive manner.

In the present specification (including the claims), when the expression such as “A configured to B,” this may include that a physically structure of A has a configuration that can execute operation B, as well as a permanent or a temporary setting/configuration of element A is configured/set to actually execute operation B. For example, when the element A is a general-purpose processor, the processor may have a hardware configuration capable of executing the operation B and may be configured to actually execute the operation B by setting the permanent or the temporary program (instructions). Moreover, when the element A is a dedicated processor, a dedicated arithmetic circuit, or the like, a circuit structure of the processor or the like may be implemented to actually execute the operation B, irrespective of whether or not control instructions and data are actually attached thereto.

In the present specification (including the claims), when a term referring to inclusion or possession (for example, “comprising/including,” “having,” or the like) is used, it is intended as an open-ended term, including the case of inclusion or possession an object other than the object indicated by the object of the term. If the object of these terms implying inclusion or possession is an expression that does not specify a quantity or suggests a singular number (an expression with a or an article), the expression should be construed as not being limited to a specific number.

In the present specification (including the claims), although when the expression such as “one or more,” “at least one,” or the like is used in some places, and the expression that does not specify a quantity or suggests a singular number (the expression with a or an article) is used elsewhere, it is not intended that this expression means “one.” In general, the expression that does not specify a quantity or suggests a singular number (the expression with a or an as article) should be interpreted as not necessarily limited to a specific number.

In the present specification, when it is stated that a particular configuration of an example results in a particular effect (advantage/result), unless there are some other reasons, it should be understood that the effect is also obtained for one or more other embodiments having the configuration. However, it should be understood that the presence or absence of such an effect generally depends on various factors, conditions, and/or states, etc., and that such an effect is not always achieved by the configuration. The effect is merely achieved by the configuration in the embodiments when various factors, conditions, and/or states, etc., are met, but the effect is not always obtained in the claimed invention that defines the configuration or a similar configuration.

In the present specification (including the claims), when the term such as “maximize/maximization” is used, this includes finding a global maximum value, finding an approximate value of the global maximum value, finding a local maximum value, and finding an approximate value of the local maximum value, should be interpreted as appropriate accordingly depending on the context in which the term is used. It also includes finding on the approximated value of these maximum values probabilistically or heuristically. Similarly, when the term such as “minimize” is used, this includes finding a global minimum value, finding an approximated value of the global minimum value, finding a local minimum value, and finding an approximated value of the local minimum value, and should be interpreted as appropriate accordingly depending on the context in which the term is used. It also includes finding the approximated value of these minimum values probabilistically or heuristically. Similarly, when the term such as “optimize” is used, this includes finding a global optimum value, finding an approximated value of the global optimum value, finding a local optimum value, and finding an approximated value of the local optimum value, and should be interpreted as appropriate accordingly depending on the context in which the term is used. It also includes finding the approximated value of these optimal values probabilistically or heuristically.

In the present specification (including claims), when a plurality of hardware performs a predetermined process, the respective hardware may cooperate to perform the predetermined process, or some hardware may perform all the predetermined process. Further, a part of the hardware may perform a part of the predetermined process, and the other hardware may perform the rest of the predetermined process. In the present specification (including claims), when an expression (including similar expressions) such as “one or more hardware perform a first process and the one or more hardware perform a second process,” or the like, is used, the hardware that perform the first process and the hardware that perform the second process may be the same hardware, or may be the different hardware. That is: the hardware that perform the first process and the hardware that perform the second process may be included in the one or more hardware. Note that, the hardware may include an electronic circuit, a device including the electronic circuit, or the like.

While certain embodiments of the present disclosure have been described in detail above, the present disclosure is not limited to the individual embodiments described above. Various additions, changes, substitutions, partial deletions, etc. are possible to the extent that they do not deviate from the conceptual idea and purpose of the present disclosure derived from the contents specified in the claims and their equivalents. For example, when numerical values or mathematical formulas are used in the description in the above-described embodiments, they are shown for illustrative purposes only and do not limit the scope of the present disclosure. Further, the order of each operation shown in the embodiments is also an example, and does not limit the scope of the present disclosure.

1. An information processing device comprising one or more processors configured to:
optimize, for a specific elementary reaction in a reaction using a catalyst including a plurality of elementary reactions, an arrangement of a promoter element in the catalyst based on activation energy acquired using a trained model; and
search for the promoter element based on the activation energy acquired using the trained model for each type of the promoter element.

2. The information processing device according to claim 1, wherein
the one or more processors are configured to:
specify an elementary reaction related to an adsorbed molecule being an elementary reaction affecting a target property in the reaction using the catalyst including the plurality of elementary reactions; and
optimize the arrangement of the promoter element for the elementary reaction specified.

3. The information processing device according to claim 2, wherein
the one or more processors are configured to:
search for, based on the acquired activation energy for each of a plurality of types of the promoter elements, the promoter element lower or higher in the activation energy than the other promoter elements.

4. The information processing device according to claim 3, wherein
the one or more processors are configured to:
repeat a reaction path search using the trained model a plurality of times with the arrangement of one or a plurality of promoter elements designated in the catalyst; and
optimize the arrangement of the promoter element lower in the activation energy than the other arrangements.

5. The information processing device according to claim 4, wherein
the one or more processors are configured to:
acquire the activation energy using the trained model in a state where a calculation initial structure of the adsorbed molecule to be used for the reaction path search is arranged at the same position.

6. The information processing device according to claim 1, wherein
the trained model is a neural network model trained by a plurality of elements.

7. The information processing device according to claim 2, wherein
the one or more processors are configured to:
set the arrangement of the promoter element to within 5 Å from a position of the adsorbed molecule.

8. The information processing device according to claim 1, wherein
the one or more processors are configured to:
optimize the arrangement of the one promoter element using a grid search.

9. The information processing device according to claim 1, wherein
the one or more processors are configured to:
optimize the arrangement of the plurality of promoter elements using at least one of Bayesian optimization and a random search.

10. The information processing device according to claim 2, wherein
the one or more processors are configured to:
arrange less than 10% of atoms in number except for an atom constituting the adsorbed molecule in an atomic structure input into the trained model, as the promoter elements.

11. The information processing device according to claim 1, wherein
the one or more processors are configured to:
arrange the promoter element with some of atoms of the catalyst replaced with the promoter element and/or the promoter element added to the atoms of the catalyst.

12. The information processing device according to claim 1, wherein
the one or more processors are configured to further:
arrange a plurality of types of the promoter elements; and
optimize a ratio of the plurality of types of promoter elements and the arrangement of each of the promoter elements.

13. The information processing device according to claim 2, wherein
the one or more processors are configured to:
specify one or a plurality of elementary reactions.

14. The information processing device according to claim 2, wherein
the one or more processors are configured to:
execute a kinetic simulation while changing a reaction rate constant of the plurality of elementary reactions;
acquire effects of the elementary reactions changed in the reaction rate constant on the target property; and
specify the elementary reaction affecting the target property based on the effect on the target property.

15. The information processing device according to claim 14, wherein
the one or more processors are configured to:
calculate a parameter of the kinetic simulation by the trained model.

16. The information processing device according to claim 1, wherein the processor is configured to:
train a model which predicts activation energy by active learning.

17. The information processing device according to claim 1, wherein
the one or more processors are configured to:
repeat deduction of a physical property value correlated with the activation energy a plurality of times using the trained model with the arrangement of one or a plurality of promoter elements designated in the catalyst; and
optimize the arrangement of the promoter element lower in the physical property value than the other arrangements.

18. The information processing device according to claim 17, wherein
the physical property value includes at least one of an intermolecular distance, an atomic charge, adsorption energy, a vibrational frequency, a d band center, and energy of a reactive intermediate.

19. The information processing device according to claim 1, wherein
the trained model is a neural network model to be used for NNP (Neural Network Potential).

20. An information processing method comprising:
optimizing, by one or more processors, for a specific elementary reaction in a reaction using a catalyst including a plurality of elementary reactions, an arrangement of a promoter element in the catalyst based on activation energy acquired using a trained model; and
searching, by the one or more processors, for the promoter element based on the activation energy acquired using the trained model for each type of the promoter element.

21. A non-transitory computer readable medium storing a program causing one or more computers to:
optimize, for a specific elementary reaction in a reaction using a catalyst including a plurality of elementary reactions, an arrangement of a promoter element in the catalyst based on activation energy acquired using a trained model; and
search for the promoter element based on the activation energy acquired using the trained model for each type of the promoter element.
